(19) United States
(12) Patent Application Publication
(10) Pub. No.: US 2018/0149425 A1
(43) Pub. Date: May 31, 2018

(54) PROCESSES FOR PRODUCING A NATURAL GAS STREAM

(71) Applicant: UOP LLC, Des Plaines, IL (US)

(72) Inventors: Timothy Oneal, Tulsa, OK (US); Qing Xu, Mount Prospect, IL (US); Derrick Oneal, Tulsa, OK (US)

(21) Appl. No.: 15/878,192

(22) Filed: Jan. 23, 2018

Related U.S. Application Data
(63) Continuation of application No. PCT/US2016/043241, filed on Jul. 21, 2016.
(60) Provisional application No. 62/196,681, filed on Jul. 24, 2015.

(51) Int. Cl. F25J 3/02; C07C 7/00; C07C 7/04

(52) U.S. Cl. CPC F25J 3/0209; C07C 7/005; C07C 7/04; F25J 3/0233; F25J 2240/02; F25J 3/0242; F25J 2200/02; F25J 2200/76; F25J 2205/04; F25J 3/0238

(57) ABSTRACT

Processes for separating a natural gas stream from a liquid hydrocarbon stream. After being cooled, a feed stream may be separated in a first separation zone, into a gaseous stream and a liquid stream. A portion of each stream may be passed to a heat transfer zone to sub cool and then passed to a second separation zone having a distillation or fractionation column. An intermediate separation zone may be used to flash a gaseous stream from the sub-cooled liquid stream. The fractionation column may be operated to include ethane in either a residue gas stream rich in methane, or in the liquid hydrocarbon stream.

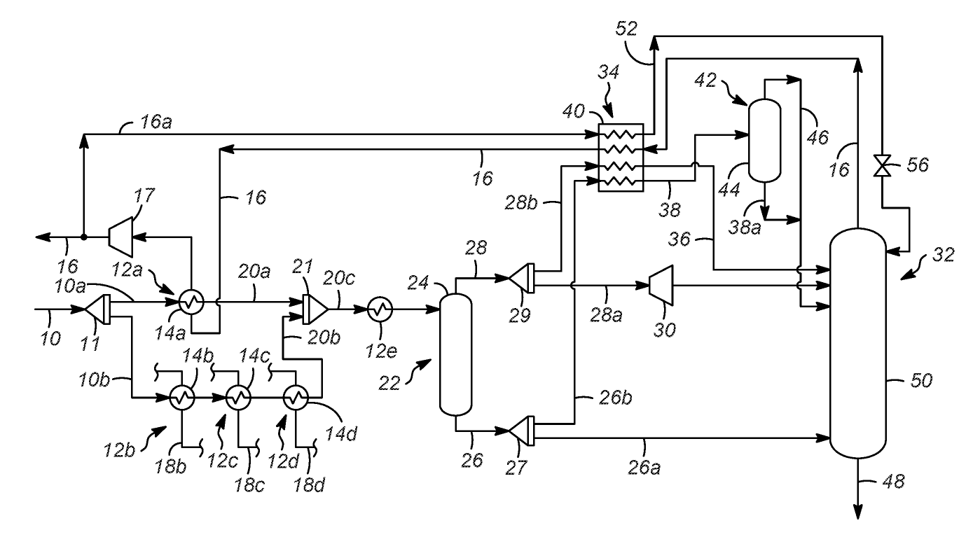

### CROSS-REFERENCE TO RELATED APPLICATIONS

This application is a Continuation of International Application No. PCT/US2016/043241 filed Jul. 21, 2016 which application claims benefit of U.S. Provisional Application No. 62/196,681 filed Jul. 24, 2015, now expired, the contents of which cited applications are hereby incorporated by reference in their entirety.

### FIELD OF THE INVENTION

This invention relates generally to processes for producing a natural gas stream, and more particularly to processes for purifying the natural gas stream by cooling the stream.

### BACKGROUND OF THE INVENTION

Natural gas from petroleum reservoirs is comprised mostly of methane but can include varying amounts of other hydrocarbons such as ethane, propane, butanes, and pentanes as well as some aromatic hydrocarbons. Additionally, natural gas may also contain non-hydrocarbon compounds, such as water, nitrogen, carbon dioxide, sulfur compounds, hydrogen sulfide, to name a few.

One type of processing plant for natural gas liquefies and separates the heavier hydrocarbon components of natural gas (ethane, propane, butanes, gasolines, etc.) from the primarily methane fraction which remains in gaseous form (residue gas). The liquefied hydrocarbons can be utilized as petrochemical feedstocks, gasoline blending components, and fuel.

In various known natural gas recovery processes, a hydrocarbon feed gas stream is cooled, for example, by heat exchange with other streams, with external sources of refrigeration such as a propane compression-refrigeration system, or both. As the gas is cooled, heavy hydrocarbons will be condensed and may be separated as a high-pressure liquid stream. After separation, the high-pressure liquids stream may be expanded to a lower pressure and then separated, for example by fractionation.

Typically, the stream may be fractionated in a distillation column such as a demethanizer column or a deethanizer column. The distillation column will provide a residue gas stream having methane and ethane or mostly methane, and a liquid stream comprising the heavier hydrocarbons.

Thus, the processes for separating and purifying the natural gas stream typically involve cooling the stream and then heating the stream.

It would be desirable for one or more processes to reduce the operating costs associated with such separation processes. It would further be desirable if such processes improved the purity of the hydrocarbon streams produced by the separation processes.

### SUMMARY OF THE INVENTION

One or more processes have been invented for separating a hydrocarbon stream into at least two streams.

In a first aspect of the present invention, the present invention may be broadly characterized as providing a process for separating a stream comprising hydrocarbons into at least two streams by: cooling a hydrocarbon stream comprising mostly methane to provide a chilled hydrocarbon stream; separating the chilled hydrocarbon stream in a first separation zone into a gaseous stream and a liquid stream; separating a first portion of the liquid stream in a second separation zone into a residue gas stream and a liquid hydrocarbon stream; cooling a second portion of the liquid stream in a heat transfer zone to provide a cooled liquid stream; separating the cooled liquid stream in the second separation zone; and, separating one or more streams comprising the gaseous stream in the second separation zone.

In one or more embodiments of the present invention, the second separation zone has an operating pressure at least 689 kPa (100 psi) lower than an operating pressure of the first separation zone.

In various embodiments of the present invention, the process further includes separating a flashed gaseous stream from the cooled liquid stream in an intermediate separation zone and separating the flashed gaseous stream in the second separation zone. It is contemplated that the intermediate separation zone has an operating pressure at least 1,724 kPa (250 psi) less than an operating pressure of the first separation zone. It is also contemplated that the second separation zone has an operating pressure at least 2,758 kPa (400 psi) lower than the operating pressure of the first separation zone.

In at least one embodiment of the present invention, the liquid hydrocarbon stream comprises ethane.

In some embodiments of the present invention, the residue gas stream comprises ethane.

In one or more embodiments of the present invention, the second portion of the liquid stream comprises between 10 to 30% (by volume) of the liquid stream from the first separation zone.

In various embodiments of the present invention, a first portion of the hydrocarbon stream is chilled by the residue gas stream.

In a second aspect of the present invention, the invention may be generally characterized as providing a process for separating a stream comprising hydrocarbons into at least two streams by: cooling a hydrocarbon stream comprising mostly methane to provide a chilled hydrocarbon stream; passing the chilled hydrocarbon stream to a first separation zone configured to separate the hydrocarbon stream into a gaseous stream and a liquid stream; splitting the liquid stream into a first portion and a second portion; passing the first portion to a second separation zone configured to provide a residue gas stream and a liquid hydrocarbon stream; passing the second portion of the liquid stream to a heat transfer zone configured to lower a temperature of the second portion of the liquid stream and to provide a cooled liquid stream; passing at least a portion of the cooled liquid stream to the second separation zone; and, passing one or more streams comprising the gaseous stream to the second separation zone.

In at least one embodiment of the present invention, the second portion of the liquid stream comprises between 10 to 30% (by volume) of the liquid stream from the first separation zone.

In various embodiments of the present invention, the process further includes splitting the hydrocarbon stream into at least two portions, chilling at least a first portion of the hydrocarbon stream with the residue gas stream, and, combining the at least two portions to form the chilled hydrocarbon stream.

In some embodiments of the present invention, the second separation zone may be operated to separate ethane into the residue gas stream, the liquid hydrocarbon stream, or both.

In some of the embodiments of the present invention, the process further includes passing the cooled liquid stream from the heat transfer zone to an intermediate separation zone configured to separate a flashed gaseous stream from the cooled liquid stream; and, passing the cooled liquid stream and the flashed gaseous stream from the intermediate separation zone to the second separation zone. It is contemplated that the intermediate separation zone has an operating pressure at least 345 kPa (50 psi) higher than an operating pressure of the second separation zone, and wherein the operating pressure of the second separation zone is at least 2,758 kPa (400 psi) less than an operating pressure of the first separation zone.

In various embodiments of the present invention, the process includes cooling a portion of the residue gas stream to provide a cooled residue gas stream and, recycling the cooled residue gas stream to the second separation zone.

In at least one embodiment of the present invention, the process also includes combining the first portion of the liquid stream from the first separation zone with the cooled liquid stream from an intermediate separation zone to form a combined liquid stream and, passing the combined liquid stream to the second separation zone.

In some embodiments of the present invention, the process includes passing a first portion of the gaseous stream from the first separation zone to an expander and, passing an expanded gas from the expander to the second separation zone. It is contemplated that the process further includes passing a second portion of the gaseous stream from the first separation zone to the heat transfer zone, cooling the second portion of the gaseous stream in the heat transfer zone to provide a cooled gaseous stream, and, passing the cooled gaseous stream from the heat exchange zone to the second separation zone.

In one or more embodiments of the present invention, the process includes combining the first portion of the liquid stream from the first separation zone with at least a portion of the cooled liquid stream, to form a combined liquid stream, and, passing the combined liquid stream to the second separation zone.

Additional aspects, embodiments, and details of the invention, all of which may be combinable in any manner, are set forth in the following detailed description of the invention.

### DETAILED DESCRIPTION OF THE DRAWING

One or more exemplary embodiments of the present invention will be described below in conjunction with the following drawing FIGURE, in which:

The FIGURE shows a process flow diagram according to one or more embodiments of the present invention.

### DETAILED DESCRIPTION OF THE INVENTION

As mentioned above, one or more processes have been invented for separating a hydrocarbon stream to produce a liquid stream and a gaseous natural gas stream. In various embodiments, a portion of a liquid stream from a low temperature separator is cooled further, for example via, cross exchange with the overhead vapors from a distillation column to sub cool the liquid. This sub-cooled liquid may then be flashed to a pressure above the operating pressure of the distillation column. This produces a very lean vapor and a sub cooled rich liquid. These two streams can either be blended with other inlet streams passed to the distillation column or introduced as their own inlets to the distillation column to enhance the separation. The current processes do not utilize the sub-cooled liquids to change the mass transfer capacity within the distillation column. It is believed that such processes will reduce the operating costs associated with the separation and purification of the natural gas stream. Additionally, such processes are also believed to improve the purity and increase the recovery of the desired components.

With these general principles in mind, one or more embodiments of the present invention will be described with the understanding that the following description is not intended to be limiting.

As shown in the FIGURE, a hydrocarbon stream 10 is provided that comprises mostly, i.e., at least 50% vol., methane that is cooled. The hydrocarbon stream 10 may also include ethane, and heavier hydrocarbons up to C10 hydrocarbons, as well hydrocarbons containing heteroatoms, such as sulfur, water, carbon dioxide and other components. Typically, the hydrocarbon stream 10 has a temperature of about 26.7 to 61.7° C. (80 to 125° F.) and a pressure of about 6,205 to 6,895 Kpa (900 to 1000 psi) (absolute).

Although not depicted as such, the hydrocarbon stream 10 may pass through a decontamination zone (not shown) to remove sulfur and other heteroatom containing compounds.

Additionally, a dehydration zone (not shown) may be used to remove any water. The decontamination zone and the dehydration zone are known in the art. For example, the decontamination zone may comprise acid gas removal by molecular sieve or amine scrubbing and dehydration by molecule sieve and/or glycol dehydration.

In the depicted embodiment, the hydrocarbon stream 10 is split into a first portion 10a and a second portion 10b by splitter 11. The split ratio of the first portion 10a to the second portion 10b may be 60:40 or 50:50, or 40:60. In order to cool the portions 10a, 10b of the hydrocarbon stream 10 to condense and separate the heavier hydrocarbons from the methane, one or more heat exchanger zones 12a, 12b, 12c, 12d may be used. In the depicted embodiment, the hydrocarbon stream 10 is first split into a first portion 10a and a second portion 10b.

As shown in the FIGURE, the first portion 10a of the hydrocarbon stream 10 may be passed to heat exchange zone 12a having a heat exchanger 14a and being cooled by a gaseous residue stream 16 (discussed below). The second portion 10b of the hydrocarbon stream 10b may be passed through at least one of the heat exchange zones 12b, 12c, 12d and pass through a heat exchanger 14b, 14c, 14d to be cooled by any number of streams 18b, 18c 18d. For example, one of the heat exchangers 14b may allow for heat exchange between the second portion 10b of the hydrocarbon stream 10 and a stream 18b that comprises a liquid hydrocarbon product stream (discussed below).

Additionally, one of the heat exchangers 14c allows for heat exchange between the second portion 10b of the hydrocarbon stream 10 and a stream 18c liquid from a separation column, such as a reboiler for a separation column. It should be appreciated that, a single heat exchange zone 12a, 12b, 12c, 12d and/or single heat exchanger 14a, 14b, 14c, 14d may be used which allows for multiple streams to exchange heat. The number and configuration of the heat exchange zones 12a, 12b, 12c, 12d and the heat exchangers 14a, 14b, 14c, 14d can be modified in any number of configurations, the specifics of which are not necessary for an understanding or practicing of the present invention.

Returning to the FIGURE, from the various heat exchange zones 12a, 12b, 12c, 12d, chilled hydrocarbon streams 20a, 20b, respectively, may be combined in mixer 21 to form a combined chilled hydrocarbon stream 20c. The combined chilled hydrocarbon stream 20c may be further cooled in another heat exchange zone 12e and then passed to a first separation zone 22. Alternatively, although not depicted as such, instead of being combined, the chilled hydrocarbon streams 20a, 20b may each be passed independently into the first separation zone 22, without or without passing through additional heat exchange zones, such as the heat exchange zone 12e shown in the FIGURE.

The first separation zone 22 preferred includes a separation vessel 24 having an operating temperature of about −12 to about −34° C. (about 10 to about −30° F.) and operating pressure preferably between about 5,516 to about 6,205 kPa (about 800 to about 900 psi). In the separation vessel 24 of the first separation zone 22, the components of the combined chilled hydrocarbon stream 20c are separated into a liquid stream 26 and a gaseous stream 28. The gaseous stream 28 may be split into two portions 28a, 28b via a splitter 29. The ratio of first portion 28a to second portion 28b of the gaseous stream 28 may be from 20:80 to 35:65. The liquid stream 26 from the first separation vessel 24 may also be separated into a first portion 26a and a second portion 26b via a splitter 27. The ratio of first portion 26a to second portion 26b of the liquid stream 26 may be 90:10, or 80:20, or 70:30.

The first portion 28a of the gaseous stream 28 may be passed to an expander 30, such as a turbo expander, to lower the pressure and then passed to a second separation zone 32 (discussed in more detail below). The first portion 26a of the liquid stream 26 from the first separation vessel 24 may also be passed to the second separation zone 32.

The second portion 28b of the gaseous stream 28 is preferably passed to a heat transfer zone 34. In the heat transfer zone 34, the second portion 28b of the gaseous stream 28 is cooled, for example via cross exchange with the residue stream 16 (discussed below) to provide a cooled gaseous stream 36. The cooled gaseous stream 36 may be passed to the second separation zone 32. The second portion 26b of the liquid stream 26 from the first separation zone 22 may also passed to the heat transfer zone 34 to cool the second portion 26b of the liquid stream 26 to provide a cooled liquid stream 38 that has been sub-cooled. As will be appreciated, the heat transfer zone 34 preferably includes a heat exchanger 40 that will cool both the second portion 26b of the liquid stream 26 and the second portion 28b of the gaseous stream 28. In the heat transfer zone 34, it is preferred that gaseous components are preferably condensed. The operating temperature of such a heat exchange zone 34 is preferably between about −46 and −73.3° C. (about −50 to −100° F.).

The cooled liquid stream 38 and the cooled gaseous stream 36 are passed to the second separation zone 32, however, in a preferred embodiment, such as the one depicted in the FIGURE, the cooled liquid stream 38 from the heat transfer zone 34 is first passed to an intermediate separation zone 42.

The intermediate separation zone 42 comprises a vessel 44 having an operating pressure about 2,070 to about 3,100 kPa (about 300 to 450 psi) and an operating temperature between about −40 to about −62° C. (about −40 to about −80° F. The vessel 44 of the intermediate separation zone 42 will separate any gaseous components in the cooled liquid stream 38 into a flashed gaseous stream 46.

Separating the gaseous components, as discussed above, will alter the mass transfer capacity of the second separation zone 32. The flashed gaseous stream 46 and a remaining portion 38a of the cooled liquid stream 38 may be passed to the second separation zone 32. Although these two streams 46, 38a are depicted as being combined and the combined stream passed to the second separation zone 32, the two streams 46, 38a may each be passed into the second separation zone 32 individually. Additionally, it is also contemplated that one or more of these streams 46, 38a is combined with any of the other streams that is passed to the second separation zone 32, such as the first portion 26a of the liquid stream 26, the first portion 28a of the gaseous stream 28 (after is has been passed through the expander 30), and the cooled gaseous stream 36. Those of ordinary skill in the art will appreciate that any number of combinations of combining streams may be used without departing from the principles of the present invention. The flashed gaseous stream 46 will typically create a top feed reflux in the second separation zone 32, and thus may be passed to the second separation zone 32 without being combined with another stream. Accordingly, the remaining portion 38a of the cooled liquid stream 38 may be combined with the first portion 26a of the liquid stream 26, the first portion 28a of the gaseous stream 28 (after is has passed through the expander 30), or be passed on its own to the second separation zone 32.

In the second separation zone 32, the various components of the streams will be separated into the residue gas stream 16 and a liquid hydrocarbon stream 48. Thus, the second separation zone 32 comprises a fractionation column 50 that separates the components by boiling point. In some embodiments of the residue gas stream 16 may include ethane. On the other hand, in some embodiments, the fractionation column 50 is operated such that ethane is in the liquid hydrocarbon stream 48. For embodiments in which the ethane is in the residue gas stream 16, the fractionation column 50 may have an inlet temperature of about −73° C. (about −100° F.) and an operating pressure between about 1,724 to about 2,413 kPa (about 250 to about 350 psi). For embodiments in which ethane is in the liquid hydrocarbon stream 48, the fractionation column 50 may have an inlet temperature of about −113° C. (about −171° F.) and an operating pressure of between about 1,379 to about 1,724 kPa (about 200 to about 250 psi).

Preferably, the second separation zone 32 has an operating pressure between about 1,379 to about 2,413 kPa (about 200 and about 350 psi) and at least about 689 kPa (about 100 psi) or more preferably at least about 2,758 kPa (about 400 psi) lower than the operating pressure of the first separation zone 22. Additionally, the intermediate separation zone 42 may have a pressure that is at least about 1,724 kPa (about 350 psi) lower than the operating pressure of the first separation zone 22, while at least about 345 pKa (about 50 psi) higher, preferably approximately about 689 kPa (about 100 psi) than the operating pressure of the second separation zone 32.

The liquid hydrocarbon product stream 48 can be used, as discussed above, as a stream 18b, 18c, 18d to cool at least a portion of the feed hydrocarbon stream 10 in one or more of the heat exchange zones 12b, 12c, 12d. Although not depicted as such, it is also contemplated that one or more side draws from separation zone 32, more specifically the fractionation column 50, are typically used as streams 18b, 18c, 18d for heat exchange zones 12b, 12c, 12d. As mentioned at the outset, the liquid hydrocarbon product stream 48 can be used as fuel, as a gasoline blending component, or in any other manner.

The residue gas stream 16, as mentioned above, may be passed to the heat transfer zone 34 to exchange heat with 28b and 26b. From the heat transfer zone 34, the residue gas stream 16 may be passed used to cool a portion of the feed hydrocarbon stream 10 in a heat exchange zone 12a. The residue gas stream 16, either comprising methane (95% molar methane and inert light compounds), or a mixture of methane and ethane (between 82 to 95% molar methane, the bulk (greater than 50% molar) of the ethane and inert light compounds) can be further processed as is known in the art.

It is also contemplated that a slip stream 16a of the residue gas stream 16, preferably after heat exchange with the first portion 10 of the hydrocarbon stream 10 in the heat exchange zone 12a and after recompression of the residue gas stream 16 in compressor 17, is cooled, for example, by heat exchange with the residue gas stream 16 to produce a cooled residue stream 52 having both liquid and vapor. The cooled residue stream 54 may be expanded through a pressure valve 56 to a pressure near that of the fractionation column 50 before being passed back to the fractionation column 50 in the second separation zone 32 typically as a top reflux. Additional modifications of the exemplary processes will be apparent to those of ordinary skill in the art.

As mentioned above, by cooling a portion of the liquid from the first separation zone, the mass balance of liquid and gas in the second separation zone can be shifted to provide for a better and more cost effective separation of the residue gas stream from the liquid hydrocarbon stream. By sub cooling a portion of the low temperature separator liquids and then flashing those liquids and separating them, the two stream can be introduced at new or mixed with existing tower feed locations in order to optimize the capture of either ethane or propane in both a rejection or recovery scenario based on desired component capture and the richness of a given gas that location may vary.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention. Furthermore, unless otherwise identified, all pressures are absolute.

### Specific Embodiments

While the following is described in conjunction with specific embodiments, it will be understood that this description is intended to illustrate and not limit the scope of the preceding description and the appended claims.

A first embodiment of the invention is a process for separating a stream comprising hydrocarbons into at least two streams, the process comprising cooling a hydrocarbon stream comprising mostly methane to provide a chilled hydrocarbon stream; separating the chilled hydrocarbon stream in a first separation zone into a gaseous stream and a liquid stream; separating a first portion of the liquid stream in a second separation zone into a residue gas stream and a liquid hydrocarbon stream; cooling a second portion of the liquid stream in a heat transfer zone to provide a cooled liquid stream; separating the cooled liquid stream in the second separation zone; and, separating one or more streams comprising the gaseous stream in the second separation zone. An embodiment of the invention is one, any or all of prior embodiments in this paragraph up through the first embodiment in this paragraph wherein the second separation zone has an operating pressure at least 689 kPa less than an operating pressure of the first separation zone. An embodiment of the invention is one, any or all of prior embodiments in this paragraph up through the first embodiment in this paragraph further comprising separating a flashed gaseous stream from the cooled liquid stream in an intermediate separation zone; and, separating the flashed gaseous stream in the second separation zone. An embodiment of the invention is one, any or all of prior embodiments in this paragraph up through the first embodiment in this paragraph wherein the intermediate separation zone has an operating pressure at least 1,724 kPa psi less than an operating pressure of the first separation zone. An embodiment of the invention is one, any or all of prior embodiments in this paragraph up through the first embodiment in this paragraph wherein the second separation zone has an operating pressure at least 2,758 kPa lower than the operating pressure of the first separation zone. An embodiment of the invention is one, any or all of prior embodiments in this paragraph up through the first embodiment in this paragraph wherein the liquid hydrocarbon stream comprises ethane. An embodiment of the invention is one, any or all of prior embodiments in this paragraph up through the first embodiment in this paragraph wherein the residue gas stream comprises ethane. An embodiment of the invention is one, any or all of prior embodiments in this paragraph up through the first embodiment in this paragraph wherein the second portion of the liquid stream comprises between 10 to 30% by volume of the liquid stream from the first separation zone. An embodiment of the invention is one, any or all of prior embodiments in this paragraph up through the first embodiment in this paragraph wherein a first portion of the hydrocarbon stream is chilled by the residue gas stream.

A second embodiment of the invention is a process for separating a stream comprising hydrocarbons into at least two streams, the process comprising cooling a hydrocarbon stream comprising mostly methane to provide a chilled hydrocarbon stream; passing the chilled hydrocarbon stream to a first separation zone configured to separate the hydrocarbon stream into a gaseous stream and a liquid stream; splitting the liquid stream into a first portion and a second portion; passing the first portion to a second separation zone configured to provide a residue gas stream and a liquid hydrocarbon stream; passing the second portion of the liquid stream to a heat transfer zone configured to lower a temperature of the second portion of the liquid stream and to provide a cooled liquid stream; passing at least a portion of the cooled liquid stream to the second separation zone; and, passing one or more streams comprising the gaseous stream to the second separation zone. An embodiment of the invention is one, any or all of prior embodiments in this paragraph up through the second embodiment in this paragraph, wherein the second portion of the liquid stream comprises between 10 to 30% by volume of the liquid stream from the first separation zone. An embodiment of the invention is one, any or all of prior embodiments in this paragraph up through the second embodiment in this paragraph further comprising splitting the hydrocarbon stream into at least two portions; and, chilling at least a first portion of the hydrocarbon stream with the residue gas stream; and, combining the at least two portions to form the chilled hydrocarbon stream. An embodiment of the invention is one, any or all of prior embodiments in this paragraph up through the second embodiment in this paragraph wherein the second separation zone may be operated to separate ethane into the residue gas stream, the liquid hydrocarbon stream, or both. An embodiment of the invention is one, any or all of prior embodiments in this paragraph up through the second embodiment in this paragraph further comprising passing the cooled liquid stream from the heat transfer zone to an intermediate separation zone configured to separate a flashed gaseous stream from the cooled liquid stream; and, passing the cooled liquid stream and the flashed gaseous stream from the intermediate separation zone to the second separation zone. An embodiment of the invention is one, any or all of prior embodiments in this paragraph up through the second embodiment in this paragraph, wherein the intermediate separation zone has an operating pressure at least 345 kPa higher than an operating pressure of the second separation zone, and wherein the operating pressure of the second separation zone is at least 2,758 kPa less than an operating pressure of the first separation zone. An embodiment of the invention is one, any or all of prior embodiments in this paragraph up through the second embodiment in this paragraph further comprising cooling a portion of the residue gas stream to provide a cooled residue gas stream; and, recycling the cooled residue gas stream to the second separation zone. An embodiment of the invention is one, any or all of prior embodiments in this paragraph up through the second embodiment in this paragraph further comprising combining the first portion of the liquid stream from the first separation zone with the cooled liquid stream from an intermediate separation zone to form a combined liquid stream; and, passing the combined liquid stream to the second separation zone. An embodiment of the invention is one, any or all of prior embodiments in this paragraph up through the second embodiment in this paragraph further comprising passing a first portion of the gaseous stream from the first separation zone to an expander; and, passing an expanded gas from the expander to the second separation zone. An embodiment of the invention is one, any or all of prior embodiments in this paragraph up through the second embodiment in this paragraph further comprising passing a second portion of the gaseous stream from the first separation zone to the heat transfer zone; cooling the second portion of the gaseous stream in the heat transfer zone to provide a cooled gaseous stream; and, passing the cooled gaseous stream from the heat transfer zone to the second separation zone. An embodiment of the invention is one, any or all of prior embodiments in this paragraph up through the second embodiment in this paragraph further comprising combining the first portion of the liquid stream from the first separation zone with at least a portion of the cooled liquid stream, to form a combined liquid stream; and, passing the combined liquid stream to the second separation zone.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for separating a stream comprising hydrocarbons into at least two streams, the process comprising:
cooling a hydrocarbon stream comprising mostly methane to provide a chilled hydrocarbon stream;
separating the chilled hydrocarbon stream in a first separation zone into a gaseous stream and a liquid stream;
separating a first portion of the liquid stream in a second separation zone into a residue gas stream and a liquid hydrocarbon stream;
cooling a second portion of the liquid stream in a heat transfer zone to provide a cooled liquid stream;
separating the cooled liquid stream in the second separation zone; and,
separating one or more streams comprising the gaseous stream in the second separation zone.

2. The process of claim 1 wherein the second separation zone has an operating pressure at least 689 kPa less than an operating pressure of the first separation zone.

3. The process of claim 1 further comprising:
separating a flashed gaseous stream from the cooled liquid stream in an intermediate separation zone; and,
separating the flashed gaseous stream in the second separation zone.

4. The process of claim 3 wherein the intermediate separation zone has an operating pressure at least 1,724 kPa psi less than an operating pressure of the first separation zone.

5. The process of claim 4 wherein the second separation zone has an operating pressure at least 2,758 kPa lower than the operating pressure of the first separation zone.

6. The process of claim 1 wherein the liquid hydrocarbon stream comprises ethane.

7. The process of claim 1 wherein the residue gas stream comprises ethane.

8. The process of claim 1 wherein the second portion of the liquid stream comprises between 10 to 30% by volume of the liquid stream from the first separation zone.

9. The process of claim 1 wherein a first portion of the hydrocarbon stream is chilled by the residue gas stream.

10. A process for separating a stream comprising hydrocarbons into at least two streams, the process comprising:
cooling a hydrocarbon stream comprising mostly methane to provide a chilled hydrocarbon stream;
passing the chilled hydrocarbon stream to a first separation zone configured to separate the hydrocarbon stream into a gaseous stream and a liquid stream; splitting the liquid stream into a first portion and a second portion;
passing the first portion to a second separation zone configured to provide a residue gas stream and a liquid hydrocarbon stream;
passing the second portion of the liquid stream to a heat transfer zone configured to lower a temperature of the second portion of the liquid stream and to provide a cooled liquid stream;
passing at least a portion of the cooled liquid stream to the second separation zone; and,
passing one or more streams comprising the gaseous stream to the second separation zone.

11. The process of claim 10, wherein the second portion of the liquid stream comprises between 10 to 30% by volume of the liquid stream from the first separation zone.

12. The process of claim 10 further comprising:
splitting the hydrocarbon stream into at least two portions; and,
chilling at least a first portion of the hydrocarbon stream with the residue gas stream; and,
combining the at least two portions to form the chilled hydrocarbon stream.

13. The process of claim 10 wherein the second separation zone may be operated to separate ethane into the residue gas stream, the liquid hydrocarbon stream, or both.

14. The process of claim 10 further comprising:
passing the cooled liquid stream from the heat transfer zone to an intermediate separation zone configured to separate a flashed gaseous stream from the cooled liquid stream; and,
passing the cooled liquid stream and the flashed gaseous stream from the intermediate separation zone to the second separation zone.

15. The process of claim 14, wherein the intermediate separation zone has an operating pressure at least 345 kPa higher than an operating pressure of the second separation zone, and wherein the operating pressure of the second separation zone is at least 2,758 kPa less than an operating pressure of the first separation zone.

16. The process of claim 10 further comprising:
cooling a portion of the residue gas stream to provide a cooled residue gas stream; and,
recycling the cooled residue gas stream to the second separation zone.

17. The process of claim 10 further comprising:
combining the first portion of the liquid stream from the first separation zone with the cooled liquid stream from an intermediate separation zone to form a combined liquid stream; and,
passing the combined liquid stream to the second separation zone.

18. The process of claim 10 further comprising:
passing a first portion of the gaseous stream from the first separation zone to an expander; and,
passing an expanded gas from the expander to the second separation zone.

19. The process of claim 18 further comprising:
passing a second portion of the gaseous stream from the first separation zone to the heat transfer zone;
cooling the second portion of the gaseous stream in the heat transfer zone to provide a cooled gaseous stream; and,
passing the cooled gaseous stream from the heat transfer zone to the second separation zone.

20. The process of claim 10 further comprising:
combining the first portion of the liquid stream from the first separation zone with at least a portion of the cooled liquid stream, to form a combined liquid stream; and,
passing the combined liquid stream to the second separation zone.
